(19) United States
(12) Patent Application Publication
(10) Pub. No.: US 2024/0363237 A1
(43) Pub. Date: Oct. 31, 2024

(54) DEVICE DATA IDENTIFICATION SYSTEM AND PROCESS

(71) Applicant: Aetion, Inc., New York, NY (US)

(72) Inventors: Kathleen A.M. Troeger, Franklin, MA (US); Wendy M. Turenne, Alexandria, VA (US)

(21) Appl. No.: 18/634,384

(22) Filed: Apr. 12, 2024

Related U.S. Application Data
(60) Provisional application No. 63/462,106, filed on Apr. 26, 2023.

(51) Int. Cl. G16H 40/60; G16H 10/60

(52) U.S. Cl. CPC G16H 40/60; G16H 10/60

(57) ABSTRACT

A system and method to execute a token generation algorithm to generate a first set of tokens based on a set of medical device data. The token generation algorithm is executed to generate a second set of tokens based on a set of patient medical data. The first set of tokens and the second set of tokens are compared to identify a subset of matching tokens. A dataset is generated including a set of records corresponding to the subset of matching tokens, where a first record of the set of records comprises a first portion of medical device data and a second portion of patient medical data. A provisioning of at least a portion of the set of records to a user system is caused.

### CROSS-REFERENCE TO RELATED APPLICATION

This application claims the benefit of U.S. Provisional Application No. 63/462,106, titled “Device Data Identification System and Method,” filed Apr. 26, 2023, which is hereby incorporated herein by reference in its entirety.

### TECHNICAL FIELD

The disclosure relates generally to a device data identification system and process, particularly systems and processes to identify information relating to communicatively connected medical devices (e.g., wearable or implantable smart medical devices) maintained in the device manufacturer's database with one or more third party datasets to identify, track, and monitor medical-related information associated with each individual medical device and/or device user.

### BACKGROUND

Medical device manufacturers have difficulty reliably identifying information associated with the manufacturer's own “smart device” (i.e., a wearable or implantable device) that is communicatively accessible (e.g., via a wired or wireless connection) within other third-party datasets including a patient's medical information. The failure to efficiently identify individual units and brands of medical devices results in more complicated analyses and reporting, classification uncertainty, impediments to innovation and optimal delivery of medical care, delays in identifying patient benefits, or patient harms related to adverse events, potential cybersecurity concerns or manufacturer safety recalls. An opportunity to address this difficulty exists for manufacturers of smart or internet connected devices.

Certain manufacturers collect and store data associated with the wearable or smart implantable devices via sensor-enabled hardware and software applications. This device data can include patient level data which is unique to a manufacturer's specific device. This information is collected, with patient consent, and aggregated into a manufacturer's database. The information can be used to monitor device performance, generate reports, and inform design decisions that guide the total product lifecycle.

Disadvantageously, the communicatively connected device is used to create proprietary databases that are unique to the respective manufacturer. At the same time, healthcare entities record information on patients under their care, but they may record general data rather than data specific to the exact device. For example, a hospital billing database may show that a pacemaker device was implanted in a patient, but not record the exact brand, model, and version of that pacemaker. As a result, a device manufacturer may be unable to identify information related to its own medical device within dataset(s) created by hospitals, healthcare networks, remote or ambulatory care providers, home care providers, or aggregated third-parties (e.g., health insurance provider datasets, datasets including electronic health records associated with one or more physician offices, etc.).

The aforementioned difficulties in identifying and differentiating one brand of device from others in the same category or class (e.g., glucose monitors, infusion pumps, dialysis machines, smart needles) occurs because many medical devices lack unique device identifiers (UDIs). Furthermore, even when present, UDIs and stock keeping units (SKU) are not systematically collected in electronic medical records (EMR) or claims-based information systems, and are unavailable without the use of complex search technologies like natural language processing (NLP) and machine learning (ML).

The ability to identify the use of a specific device in electronic health record databases is critical to support data driven decision making across a number of stakeholders such as understanding the use and impact of a medical device supports product development and the ability to meet regulatory requirements for safety and monitoring (device manufacturers); greater insight into health outcomes to strengthen regulatory oversight of medical devices (regulatory bodies); and understand impact on health care outcomes, utilization and value (for payers, providers, and patients).

### BRIEF DESCRIPTION OF THE FIGURES

The present disclosure will be understood more fully from the detailed description given below and from the accompanying drawings of various implementations of the disclosure.

FIG. 1 illustrates an example device data identification system communicatively coupled to one or more medical device manufacturer systems and one or patient medical data systems, according to one or more embodiments of the present disclosure.

FIG. 2 illustrates example phases of functionality executed by an example device data identification system, according to embodiments of the present disclosure.

FIG. 3 illustrates an example data structure including a set of example records including medical device data, according to one or more embodiments of the present disclosure.

FIG. 4 illustrates an example data structure including medical device tokens generated based on medical device data maintained in a medical device manufacturer dataset, according to one or more embodiments of the present disclosure.

FIG. 5 illustrates an example data structure including a medical device dataset with patient medical data token information generated by the device data identification system, according to one or more embodiments of the present disclosure.

FIG. 6 illustrates an example data structure generated by a device data identification system including example medical device tokens compared to example patient medical data tokens, with indications of identified matches, according to one or more embodiments of the present disclosure.

FIG. 7 illustrates an example data structure including records associated with medical device tokens and patient medical data tokens identified as matching by a device data identification system, according to one or more embodiments of the present disclosure.

FIG. 8 is an example flow diagram relating to a process executed by a device data identification system, according to one or more embodiments of the present disclosure.

FIG. 9 is a block diagram of an example computer system in which implementations of the present disclosure can operate.

### DETAILED DESCRIPTION

Embodiments of the present disclosure address the above-identified problems and other deficiencies with identifying data associated with “smart” medical devices by providing a device data identification system and process for accurately identifying data related to communicatively connected smart devices among various patient records stored in large commercial or similarly aggregated general purpose datasets that are not configured for the manufacturer's medical devices.

According to embodiments, the device data identification system and process tracks and identifies “smart device” data collected by manufacturers, via wearable or implantable devices and sensor enabled hardware and software applications to provide patient level data that is unique to a manufacturer's specific device. In an embodiment, the medical device data is collected, with patient consent, and aggregated into a manufacturer's database. The medical device data is used to monitor device performance, generate reports, and guide or inform design decisions. The device is connected via a wired or wireless connection capable of electronic communications and enables the manufacturer's proprietary dataset (also referred to as the “manufacturer dataset”) to provide information that facilitates the isolation of data associated with each unique device (e.g., information identifying the medical device and related medical information) from other data and data records associated with a large number of patients and devices stored in one or more different health data systems (herein referred to as “medical record datasets”). Example medical record datasets can include datasets storing medical records (e.g., a collection of health-related data) associated with a health care provider (e.g., a hospital, a health care network, remote or ambulatory care providers, home health care providers), an aggregated healthcare-related dataset (e.g., health insurance providers, electronic health records associated with one or more physician offices, etc.), etc.

Advantageously, the device data identification system and process of the present disclosure enables a medical device manufacturer or other entity to isolate medical device-specific data stored in one or more other (i.e., independent) medical record datasets. According to embodiments, the medical devices can include any internet-enabled or wireless remote communicating (e.g., a 5G network) medical imaging equipment, laboratory and capital equipment, medication, and care delivery systems, including mobile robots and drones, as well as wearable and implantable medical devices. According to embodiments, the device data identification system and process causes execution of a token generation algorithm or process to generate a first set of tokens corresponding to a first set of data stored in a manufacturer database (herein the “manufacturer device data” or “device data”). The manufacturer data can include information relating to a manufacturer-specific medical device (e.g., a device identifier), patient information (e.g., patient identifier, patient address information, patient date of birth, patient name, etc.), etc. In an embodiment, a unique token (also referred to as a “device token” or “medical device token”) can be generated based on the manufacturer data to represent each of the manufacturer's medical devices.

According to embodiments, the device data identification system and process causes execution of the token generation algorithm or process to generate a second set of tokens corresponding to a second set of data (also referred to as “patient medical data”) stored in one or more medical record datasets (also referred to as a “patient medical record source” or “health data system”). The patient medical data can include information relating to patient data including data associated with the manufacturer-specific medical devices (e.g., patients associated with the smart medical devices), such as, for example, patient information (e.g., patient identifier, patient address information, patient date of birth, patient name, etc.), and the corresponding medical device. In an embodiment, a schema of a medical device identifier stored in the manufacturer dataset is different from a schema of a medical device identifier stored in the medical record dataset. In an embodiment, a unique token (also referred to as a “patient medical data token” or a “medical data token”) can be generated based on an individual medical record stored in the medical record dataset of a health data system.

According to embodiments, the device data identification system and process compares the first set of device tokens to the second set of patient medical data tokens to identify a subset of matches (i.e., a subset of matching tokens). In an embodiment, a match is identified by generating a similarity score based on a comparison of the respective device tokens to each respective patient medical data token. In an embodiment, a match is identified if a first condition is satisfied, where a similarity score for a token pair (e.g., a similarity score for device token ABC and patient medical data token 123) is greater than or equal to a similarity threshold level (e.g., an integer in a range of 0 to 100, where 0 represents a low similarity score and 100 represents a high similarity score).

According to embodiments, the device data identification system and process generates a dataset (herein the “matching token dataset” or “merged token dataset”) including a series of records corresponding to the matching pairs of device tokens and patient medical data tokens. For example, the matching token dataset includes a record or entry including first data from a first device token (e.g., device token ABC) and second data from a first patient medical data token (e.g., patient medical data token 123) when the similarity score corresponding to the comparison of the first device token and the first patient token is greater than or equal to the similarity threshold level.

According to embodiments, the device data identification system and process provides access to one or more end-users (i.e. an end-user system or user system) to enable interaction and the execution of one or more actions with respect to the matching token dataset. Example actions include searching, querying, analyzing, generating reports, filtering, etc.

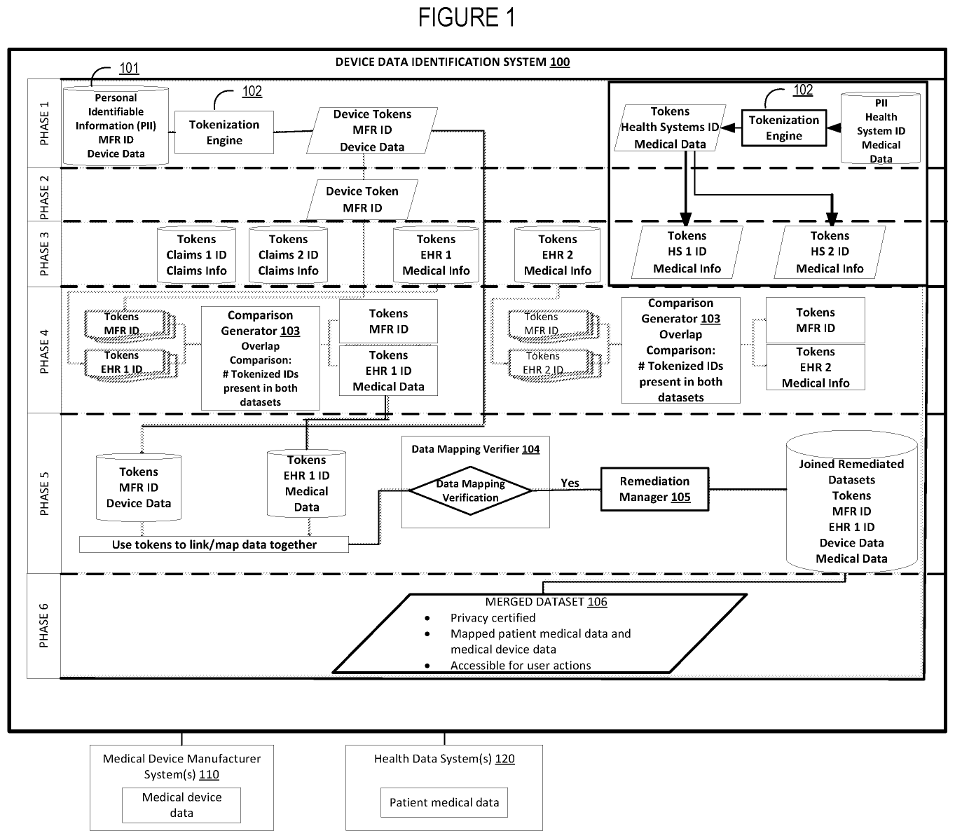

FIG. 1 illustrates an example computing environment including a device data identification system 100 executing one or more device data identification processes, as described in detail herein. The device data identification system 100 is communicatively coupled to one or more medical device manufacturer systems 110 and one or more patient medical data systems 120. In an embodiment, the device data identification system 100 interrogates the information stored in the medical device manufacturer's dataset and creates tokenized information (device tokens) corresponding to a patient receiving and/or utilizing a specific device of the manufacturer.

In an embodiment, the device data identification system 100 causes the generation of tokens for records in one or more medical record datasets maintained by one or more health data systems 120 (e.g., an insurance system, a network-level emergency medical record (EMR) system, etc.) that includes patient-level information for patients utilizing devices associated with one or more device manufacturers. In an embodiment, the manufacturer dataset of a first manufacturer's tokens (also referred to as a “sponsor manufacturer”) can be compared by the device data identification system 100 to the tokens generated based on the patient medical data stored by one or more health data systems 120 to identify patient records associated with unique medical devices, to advantageously identify and isolate the target medical device data (i.e., medical device data associated with the sponsor manufacturer) from other records stored in the one or more health record dataset of the one or more health data systems 120.

Furthermore, the device data identification system 100 maps or links the exact source/manufacturer of a medical device to a particular patient. The device data identification system 100 further enables the merging or linking of data from the device token and the patient medical data tokens. The generation of a merged or linked dataset (herein a “merged dataset”) including a transformed set of data fields allows a manufacturer system 110 to monitor, track, and manager the performance of a particular medical device in the context of additional aspects of one or more of the patient's medical records in a deidentified way.

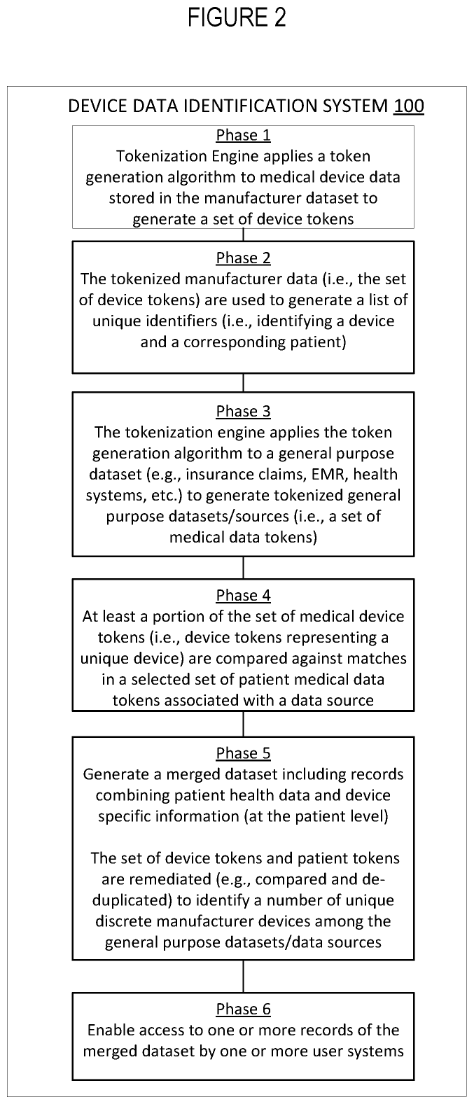

As illustrated in FIGS. 1 and 2, the device data identification system 100 executes a token-based multi-phase process (e.g., phase 1, phase 2, phase 3 . . . phase 6) to generate a merged dataset including identified medical device data that can be accessed by one or more user systems, according to embodiments. In embodiments, as illustrated in FIG. 1, the device data identification system 100 employs a “reverse” tokenization process that begins with a review of a manufacturer's proprietary dataset and compares that information to tokens in one or more patient medical record datasets for the purpose of isolating a source or brand (e.g., manufacturer) of a medical device and linking the identified information with one or more other components of the one or more medical records to formulate a merged record stored in a merged dataset.

According to embodiments, the reverse tokenization process described herein changes the functionality and utility of conventional tokenization methodology, enabling it to identify discrete medical devices from one or more general purpose medical record datasets. Advantageously, the process improves accuracy by allowing the manufacturer to accurately and efficiently identify medical information and health outcomes specifically related to a target or particular medical device. In addition, the process executed by the device data identification system 100 initiates a tokenization of the manufacturer's proprietary information and compares that tokenized data with tokened data generated based on one or more medical record datasets to provide an end-user (e.g., a user associated with the manufacturer system 110) with the advantage of being able to reliably identify medical information linked to its device, without the use of time-consuming natural language processing.

Further advantages are realized by the device data identification system enabling an increase in both the speed and accuracy of isolating a unique medical device (e.g., a smart medical device), understand the medical device's performance, and compare the medical device to others in a corresponding device class. The resulting tokenized and combined database allows manufacturers, regulatory bodies and other invested stakeholders to execute analyses to understand performance, safety, and effectiveness of specific devices as well utilize the native information generated by the device to improve patient care.

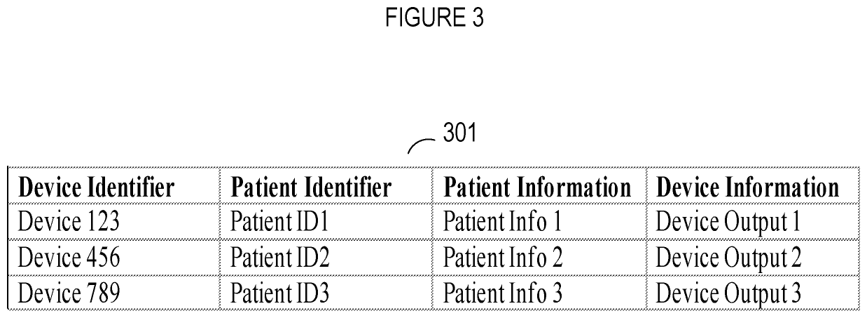

FIG. 1 and FIG. 2 provide details and descriptions of the functions, operations, workflows, steps, sub-processes, etc. performed by the device data identification system 100 during the multi-phase process to generate the merged dataset 106. In an embodiment, during phase 1, the device data identification system 100 causes execution of a tokenization engine configured to implement one or more tokenization algorithms to encrypt and encode personal identifiable information (PII) for each patient receiving and/or utilizing each medical device stored in a manufacturer dataset including device data maintained by a manufacturer system 110, to create a list of unique identifiers that enable medical device records to be associated with patient specific information. An example data structure 301 including a manufacturer dataset including records relating to device data is shown in FIG. 3.

In an embodiment, phase 1 of the multi-phase process is executed to efficiently identify a medical device, or type of medical device by creating tokens (i.e., device tokens) that enable the recognition of a particular medical device in a similarly tokenized third party (or other aggregated) data set containing patient level medical information, as shown in FIGS. 1 and 2.

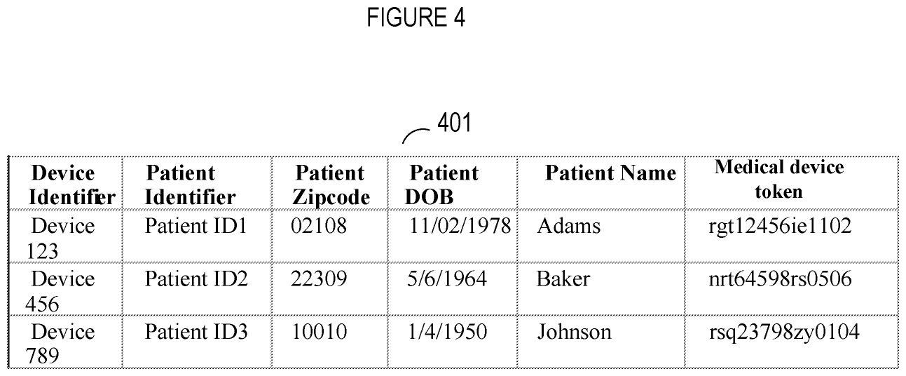

According to embodiments, in phase 2 of the process, as illustrated in FIGS. 1 and 2, the device tokens are used to generate the corresponding device and patient ID list. In an embodiment, during phase 2, the device data identification system 100 uses the device tokens to generate a list of unique identifiers, as shown in FIGS. 1 and 2. The list captures patient identifiers associated with the device user and generates a master list of device data at the patient level of detail, which can later be compared against a medical record dataset (e.g., a third party dataset). An example data structure 401 including generated device tokens and the corresponding device-token based patient identifier list is illustrated in FIG. 4.

As illustrated in FIGS. 1 and 2, according to embodiments, during phase 3 of the process, the device data identification system 100 generates tokens (i.e., patient medical data tokens) based on the patient medical data stored in one or more medical record datasets. In an embodiment, during phase 3, the device data identification system 100 causes the tokenization engine (employing a same tokenization algorithm as applied in phases 1 and 2) to generate the patient medical data tokens based on the patient medical data of one or more health data systems 120.

As shown in FIGS. 1 and 2, during phase 4, the manufacturer's set of tokens (i.e., device tokens which each represent a unique device) is compared to at least a portion of the set of patient medical data tokens. In an embodiment, the device data identification system can employ a probabilistic matching process to determine a likelihood of similarity (i.e., a similarity score) between the two token sets (i.e., the device tokens and the patient medical data tokens) and assign matches/scores. In an embodiment, token pairs with similarity scores greater than a predetermined threshold are identified as matches. This permits pairing unique devices with health history, progress, and outcomes. In an embodiment, only the medical device token and the patient medical data token are compared to one another (e.g., using a probabilistic matching process) to identify the subset of matching tokens.

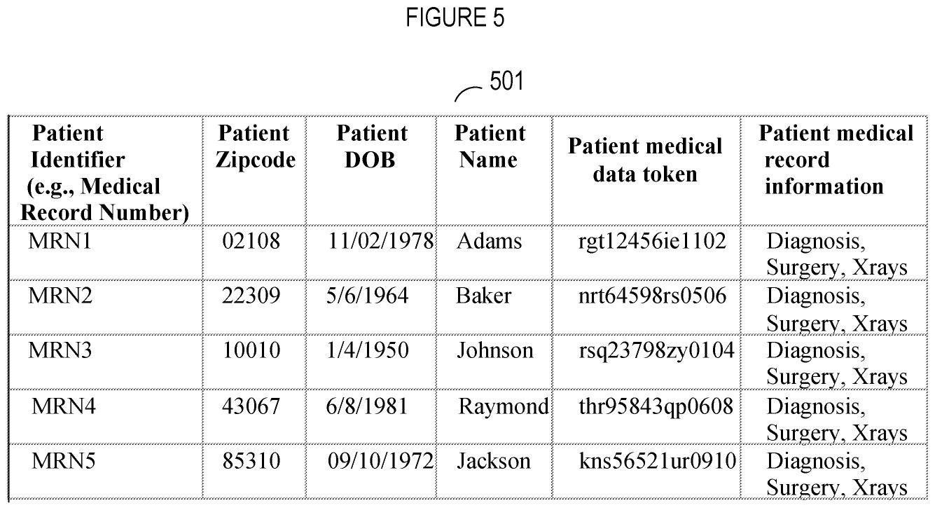

According to embodiments, the patient medical data tokens can be based on various different data fields which offer a number of options for the specific data elements that are used to generate the medical device tokens. FIG. 5 illustrates an example data structure 501 including a medical device dataset with patient medical data token information generated by the device data identification system of the present disclosure.

According to embodiments, the selection of these options determines the specificity and sensitivity of the ultimate match between two data sources. When tokenizing disparate datasets, a common tokenization scheme can be applied to the different datasets (i.e., the medical device dataset and the medical record dataset(s)) in phases 1 through 3 to generate a token for a given patient (i.e., a patient medical data token) and a medical device token that have the same schema/format, to enable efficient comparison thereof. In an embodiment, an entire token can be used for comparison and matching, and multiple token schemas can be applied and compared.

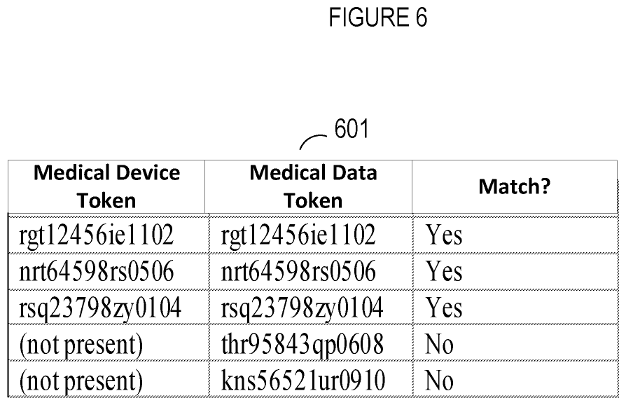

According to embodiments, as shown in FIGS. 1 and 2, during phase 4, the first set of tokens corresponding to the manufacturer dataset and the second set of tokens corresponding to the medical record dataset can be compared to identify which patient medical data records and medical device records are present in both dataset sources (i.e., the manufacturer dataset and the medical record dataset). FIG. 6 illustrates an example data structure 601 generated by the device data identification system 100 including example medical device tokens compared to example patient medical data tokens, with indications of identified matches.

According to embodiments, as shown in FIGS. 1 and 2, during phase 5 of the process, once the existence of patients and their corresponding device records is confirmed in each of the disparate data sources, both datasets are sent to a deduplication engine (e.g., a trusted broker engine or system) configured to deduplicate and merge the two data sources to generate a single merged dataset 106 that combines patient health information and the device specific information at the patient level. In an embodiment, the generated merged dataset can be reviewed to assess privacy risks (e.g., a HIPAA expert determination). In an embodiment, during phase 5, a remediation manager 105 performs one or more remediation actions (e.g., de-duplication actions) with respect to the subset of matched tokens (e.g., matching medical device tokens and patient medical data tokens) to create one or more joined remediated datasets.

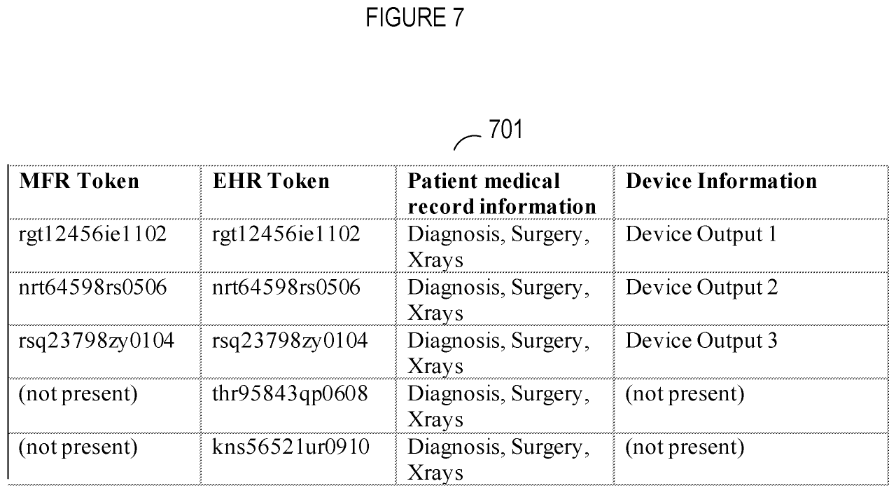

In an embodiment, as shown in FIGS. 1 and 2, in phase 6, the device data identification system 100 enables access to the merged dataset 106 by one or more end-users (e.g., a user of the manufacturer system 110) for use in performing one or more actions. In an embodiment, the device data identification system 100 causes at least a portion of the merged dataset 106 to be provisioned to one or more user systems or enables access to the merged dataset 106 (e.g., via a secure portal) to a user system. Example actions that may be performed using the merged dataset 106 include, but are not limited to, a comparison of the actual healthcare experience and outcomes associated with specific medical devices. In an embodiment, the merged dataset 106 can be used to assess whether an event (e.g., a medical event) is related to specific hardware of a medical device, software of a medical device, procedures related to the use of the medical device, manufacturing history, patient specific characteristics, etc. FIG. 7 illustrates an example data structure 701 of a tokenized merged dataset generated by the device data identification system 100, according to embodiments of the present disclosure.

According to embodiments, the process described above enables efficient and accurate isolation of internet-connected, remote-communicating medical devices and associated longitudinal medical information to outcomes specific to the class or brand of medical device. According to embodiments, the process can be executed with respect to internet-enabled medical imaging equipment, laboratory and capital equipment, medication, and care delivery systems, including mobile robots and drones as well as wearable and implantable medical devices.

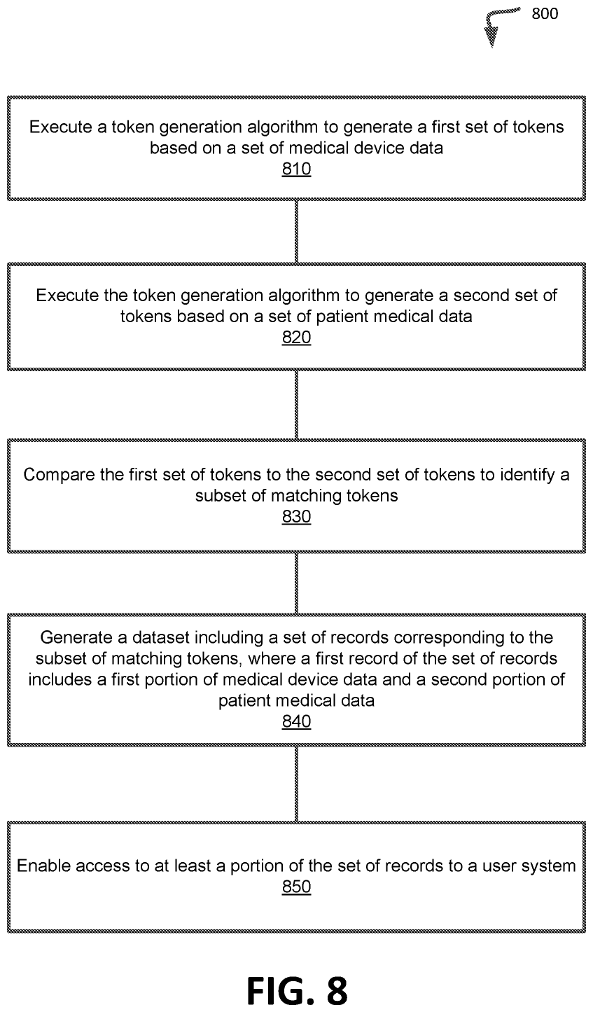

FIG. 8 illustrates a flow diagram illustrating an example process 800 including steps performed by a device data identification system (e.g., device data identification system 100 of FIG. 1) to generate a set of records corresponding to merged tokenized data sourced from multiple different datasets (e.g., a device manufacturer database and one or more patient patient medical data databases), according to embodiments of the present disclosure. The process 800 can be performed by processing logic that can include hardware (e.g., processing device, circuitry, dedicated logic, programmable logic, microcode, hardware of a device, integrated circuit, etc.), software (e.g., instructions run or executed on a processing device), or a combination thereof. Although shown in a particular sequence or order, unless otherwise specified, the order of the processes can be modified. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment. Other process flows are possible.

In operation 810, the processing logic (e.g., processing logic of the device data identification system 100 of FIGS. 1 and 2) executes a token generation algorithm to generate a first set of tokens based on a set of medical device data. In an embodiment, the medical device data is stored and maintained in one or more databases associated with a medical device manufacturer. In an embodiment, any suitable token generation algorithm may be employed to generate the first set of tokens.

In operation 820, the processing logic executes the token generation algorithm to generate a second set of tokens based on a set of patient medical data. In an embodiment, the patient medical data is stored and maintained in one or more databases associated with one or more health or medical data systems. In an embodiment, the same token generation algorithm is used to generate the first set of tokens (i.e., medical device tokens) and the second set of tokens (i.e., patient medical data tokens).

In operation 830, the processing logic compare the first set of tokens to the second set of tokens to identify a subset of matching tokens. In an embodiment, the comparison determines a likelihood of similarity (i.e., a similarity score) between the two token sets (i.e., the device tokens and the patient medical data tokens) and assign matches/scores. In an embodiment, the likelihood of similarity is based on a comparison of only the first set of tokens and the second set of tokens.

In an embodiment, each pair of tokens (e.g., a first medical device token and a first patient medical data token) is assigned a similarity score and that score is compared to a threshold score to determine if a condition is satisfied. In an embodiment, the condition is satisfied if the similarity score is greater than the threshold score. In an embodiment, if the condition is satisfied, then the particular token pair (first medical device token and the first patient medical data token) is included in the subset of matching tokens.

In operation 840, the processing logic generates a dataset including a set of records corresponding to the subset of matching tokens, where a first record of the set of records includes a first portion of medical device data and a second portion of patient medical data.

In operation 850, the processing logic enables access to at least a portion of the set of records to a user system. In an embodiment, the processing logic enables access by causing at least a portion of the records to be provisioned or transmitted to one or user systems. In an embodiment, the processing logic enables access by providing a portal or interface via which a user system can access the at least the portion of the set of records. In an embodiment, the user system can access and use the records to perform one or more actions (e.g., searching, querying, analyzing, generating reports, filtering, etc.).

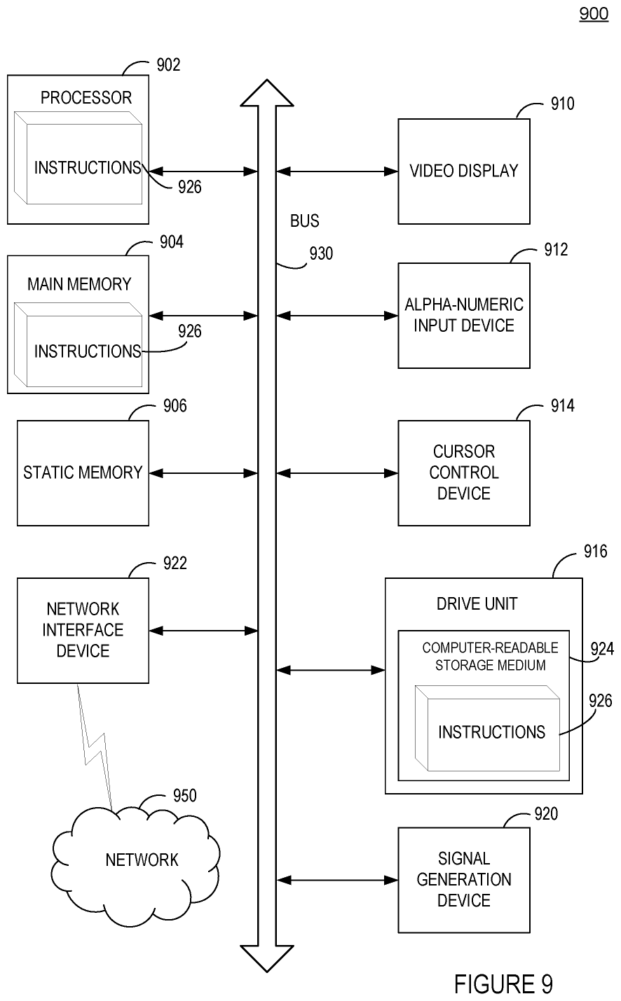

FIG. 9 is a block diagram illustrating a machine in the exemplary form of a computer system 900 in accordance with various implementations. Within the computer system 900 is a set of instructions for causing the machine to perform one or more of the device data identification processes and methodologies discussed herein. In alternative embodiments, the machine may be connected (e.g., networked) to other machines in a LAN, an intranet, an extranet, or the Internet. The machine can operate in the capacity of a server or a client machine (e.g., a computing device executing instructions associated with the device data identification system 100 and related processes) in a client-server network environment, or as a peer machine in a peer-to-peer (or distributed) network environment. The machine may be a personal computer (PC), a tablet PC, a console device or set-top box (STB), a Personal Digital Assistant (PDA), a cellular telephone, a web appliance, a server, a network router, switch or bridge, or a machine capable of executing a set of instructions (sequential or otherwise) that specify actions to be taken by that machine. Further, while only a single machine is illustrated, the term “machine” shall also be taken to include a collection of machines (e.g., computers) that individually or jointly execute a set (or multiple sets) of instructions to perform one or more of the methodologies discussed herein.

The computer system 900 includes a processing device 902 (e.g., a processor), a main memory 904 (e.g., read-only memory (ROM), flash memory, dynamic random access memory (DRAM) such as synchronous DRAM (SDRAM) or DRAM (RDRAM), etc.), a static memory 906 (e.g., flash memory, static random access memory (SRAM), etc.), and a secondary memory (e.g., a data storage device in the form of a drive unit 916, which may include fixed or removable computer-readable storage medium), which communicate with each other via a bus 930.

Processing device 902 represents one or more general-purpose processors such as a microprocessor, central processing unit, or the like. More particularly, the processing device 902 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 902 may also be one or more special-purpose processing devices such as an application specific integrated circuit (ASIC), a field programmable gate array (FPGA), a digital signal processor (DSP), network processor, or the like. Processing device 902 is configured to execute modules of the device data identification system (e.g., device data identification system 100 of FIGS. 1 and 2) for performing the operations and steps discussed herein. For example, in one embodiment, the process flows illustrated in FIGS. 1 and 2 may be performed by one or more of the modules.

The processing device 902 may be capable of executing program instructions, codes, binary instructions and the like. The processor may include memory that stores methods, codes, instructions and programs as described herein and elsewhere. The processor may access a computer-readable storage medium through an interface that may store methods, codes, and instructions as described herein and elsewhere. The computer-readable storage medium associated with the processor for storing methods, programs, codes, program instructions or other type of instructions capable of being executed by the computing or processing device may include but may not be limited to one or more floppy diskettes, optical disks, Compact Disc, Read-Only Memory (CD-ROMs), and magneto-optical disks, Read-Only Memory (ROMs), Random Access Memory (RAM), Erasable Programmable Read-Only memory (EPROM), Electrically Erasable Programmable Read-Only Memory (EEPROM), magnetic or optical cards, flash memory, or the like.

The computer system 900 may further include a network interface device 922. The computer system 900 also may include a video display unit 910 (e.g., a liquid crystal display (LCD) or a cathode ray tube (CRT)) connected to the computer system 900 through a graphics port and graphics chip-set, an alphanumeric input device 912 (e.g., a keyboard), a cursor control device 914 (e.g., a mouse), and a signal generation device 920 (e.g., a speaker).

The drive unit 916 or secondary memory may include a machine-readable storage medium (or more specifically a non-transitory computer-readable storage medium) 924 on which is stored one or more sets of instructions 926 embodying one or more of the methodologies or functions described herein with respect to the device data identification system. The sets of instructions 926 may also reside, completely or at least partially, within the main memory 904 and/or within the processor 902 during execution thereof by the computer system 900, the main memory 904 and the processing device 902 also constituting machine-readable storage media. The sets of instructions 926 may further be transmitted or received over a network 950 via the network interface device 922.

The non-transitory computer-readable storage medium 924 may also be used to store the modules of the device data identification system (e.g., the device data identification system 100 of FIGS. 1 and 2) persistently. While the computer-readable storage medium 924 is shown in as a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include a medium that is capable of storing or encoding a set of instructions for execution by the machine and that cause the machine to perform one or more of the methodologies of the present invention. The term “computer-readable storage medium” shall accordingly be taken to include, but not be limited to, solid-state memories, and optical and magnetic media.

The computing system 900, components and other features described herein can be implemented as discrete hardware components or integrated in the functionality of hardware components such as ASICS, FPGAs, DSPs or similar devices. In addition, aspects of the computing system 500 can be implemented as firmware or functional circuitry within hardware devices. Further, the computing system 900 can be implemented in a combination hardware devices and software components. For example, the functionality of this computing system 900 can exist in a fewer or greater number of modules than what is shown, with such modules residing at one or more computing devices that may be geographically dispersed. The modules may be operable in conjunction with network 950 from which it may receive and provide relevant information.

Some portions of the above-disclosure are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the above description, numerous details are set forth. It will be apparent, however, to one skilled in the art, that the present disclosure may be practiced without these specific details. In some instances, well-known structures and devices are shown in block diagram form, rather than in detail, in order to avoid obscuring the present invention. In the foregoing, implementations of the present disclosure have been described with reference to specific example implementations thereof. The specification and drawings are to be regarded in an illustrative sense rather than a restrictive sense. It will be evident various modifications and combinations of the features discussed above can be utilized without departing from the disclosure as set forth in the following claims.

The methods and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear as set forth in the description below. In addition, the scope of the disclosure is not limited to any particular programming language. It will be appreciated that a variety of programming languages may be used to implement the teachings of the disclosure.

It is to be understood that the above description is intended to be illustrative, and not restrictive. Many other embodiment examples will be apparent to those of skill in the art upon reading and understanding the above description. Although the disclosure describes specific examples, it will be recognized that the systems and methods of the disclosure are not limited to the examples described herein, but may be practiced with modifications within the scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method comprising:
executing, by a processing device, a token generation algorithm to generate a first set of tokens based on a set of medical device data;
executing the token generation algorithm to generate a second set of tokens based on a set of patient medical data;
comparing the first set of tokens and the second set of tokens to identify a subset of matching tokens;
generating a dataset including a set of records corresponding to the subset of matching tokens, wherein a first record of the set of records comprises a first portion of medical device data and a second portion of patient medical data; and
enabling access to at least a portion of the set of records to a user system.

2. The method of claim 1, further comprising generating a similarity score based on a comparison of a first token of the first set of tokens and a second token of the second set of tokens.

3. The method of claim 2, further comprising in response to determining that a condition is satisfied based on the similarity score, including the first token and the second token in the subset of matching tokens.

4. The method of claim 1, wherein the set of medical device data comprises a first medical device identifier identifying a first medical device; and wherein the set of patient medical data comprises a second medical device identifier identifying the first medical device.

5. The method of claim 4, wherein the first medical device identifier is associated with a first schema and the second medical device identifier is associated with a second schema, and wherein the first schema is different than the second schema.

6. The method of claim 1, wherein each record of the set of records comprises first information identifying a medical device and second information identifying a patient associated with the medical device.

7. The method of claim 1, wherein the first portion of medical device data is extracted from a first token of the first set of tokens; and wherein the second portion of patient medical data is extracted from a second token of the second set of tokens.

8. A system comprising:
a memory to store instructions; and
a processing device operatively coupled to the memory, the processing device to execute the instructions to perform operations comprising:
executing a token generation algorithm to generate a first set of tokens based on a set of medical device data;
executing the token generation algorithm to generate a second set of tokens based on a set of patient medical data;
comparing the first set of tokens and the second set of tokens to identify a subset of matching tokens;
generating a dataset including a set of records corresponding to the subset of matching tokens, wherein a first record of the set of records comprises a first portion of medical device data and a second portion of patient medical data; and
enabling access to at least a portion of the set of records to a user system.

9. The system of claim 8, the operations further comprising generating a similarity score based on a comparison of a first token of the first set of tokens and a second token of the second set of tokens.

10. The system of claim 9, the operations further comprising in response to determining that a condition is satisfied based on the similarity score, including the first token and the second token in the subset of matching tokens.

11. The system of claim 8, wherein the set of medical device data comprises a first medical device identifier identifying a first medical device; and wherein the set of patient medical data comprises a second medical device identifier identifying the first medical device.

12. The system of claim 11, wherein the first medical device identifier is associated with a first schema and the second medical device identifier is associated with a second schema, and wherein the first schema is different than the second schema.

13. The system of claim 8, wherein each record of the set of records comprises first information identifying a medical device and second information identifying a patient associated with the medical device.

14. The system of claim 8, wherein the first portion of medical device data is extracted from a first token of the first set of tokens; and wherein the second portion of patient medical data is extracted from a second token of the second set of tokens.

15. A non-transitory computer readable storage medium having instructions that, if executed by a processing device, cause the processing device to perform operations comprising:
executing a token generation algorithm to generate a first set of tokens based on a set of medical device data;
executing the token generation algorithm to generate a second set of tokens based on a set of patient medical data;
comparing the first set of tokens and the second set of tokens to identify a subset of matching tokens;
generating a dataset including a set of records corresponding to the subset of matching tokens, wherein a first record of the set of records comprises a first portion of medical device data and a second portion of patient medical data; and
enabling access to at least a portion of the set of records to a user system.

16. The non-transitory computer readable storage medium of claim 15, the operations further comprising generating a similarity score based on a comparison of a first token of the first set of tokens and a second token of the second set of tokens.

17. The non-transitory computer readable storage medium of claim 16, the operations further comprising in response to determining that a condition is satisfied based on the similarity score, including the first token and the second token in the subset of matching tokens.

18. The non-transitory computer readable storage medium of claim 15, wherein the set of medical device data comprises a first medical device identifier identifying a first medical device; and wherein the set of patient medical data comprises a second medical device identifier identifying the first medical device; and wherein the first medical device identifier is associated with a first schema and the second medical device identifier is associated with a second schema, wherein the first schema is different than the second schema.

19. The non-transitory computer readable storage medium of claim 15, wherein each record of the set of records comprises first information identifying a medical device and second information identifying a patient associated with the medical device.

20. The non-transitory computer readable storage medium of claim 15, wherein the first portion of medical device data is extracted from a first token of the first set of tokens; and wherein the second portion of patient medical data is extracted from a second token of the second set of tokens.
